(19) United States
(12) Patent Application Publication
(10) Pub. No.: US 2018/0049988 A1
(43) Pub. Date: Feb. 22, 2018

(54) SUSTAINED LOCAL DELIVERY OF DRUGS INCLUDING ANTIBIOTICS

(71) Applicant: Eupraxia Pharmaceuticals Inc., Victoria (CA)

(72) Inventors: Rafi Chapanian, Victoria (CA); James A. Helliwell, Victoria (CA); Amanda Malone, Victoria (CA); Vik Peck, Victoria (CA); Troy Loss, Victoria (CA); Azadeh Goudarzi, Victoria (CA)

(21) Appl. No.: 15/679,546

(22) Filed: Aug. 17, 2017

Related U.S. Application Data
(60) Provisional application No. 62/376,204, filed on Aug. 17, 2016.

(51) Int. Cl. A61K 9/28; B32B 27/04; B32B 27/08

(52) U.S. Cl. CPC A61K 9/28; B29C 37/00; B32B 27/08; B32B 27/04

(57) ABSTRACT

Described herein are extended release composite film comprising a drug-loaded membrane coated with porous coatings that is capable of localized release of drug (e.g., antibiotics) for surgical prophylaxis.

### CROSS-REFERENCES TO RELATED APPLICATIONS

This application claims the benefit under 35 U.S.C. § 119(e) of U.S. Provisional Patent Application No. 62/376,204, filed Aug. 17, 2016, which application is incorporated herein by reference in its entirety.

### BACKGROUND

### Technical Field

This disclosure relates to sustained release of active pharmaceutical agents (API) such as antibiotics and methods of using the same, including for surgical prophylaxis.

### Description of the Related Art

Surgical site infections (SSI) are infections related to an operative procedure that occurs at or near the surgical incision within 30 days of the procedure or within 90 days if prosthetic material is implanted at surgery. SSIs are often localized to the incision site but can also extend into deeper adjacent tissue or structures.

Surgical site infections typically develop due to contaminated and dirty wounds such as penetrating trauma, chronic wounds, and entry into biliary or genitourinary tract in the presence of infected bile or urine, preoperative perforation of respiratory; gastrointestinal; biliary or genitourinary tract. SSI can also happen in clean operations where a prosthetic device is inserted.

Current norm of treating SSI is by intravenous (IV) injection of antibiotics. However, IV injections in many cases are unable to provide sufficiently high concentrations of antibiotics at surgical sites. In addition, IV injections of antibiotics can cause development of antibiotic-resistant bacteria.

### BRIEF SUMMARY

Described herein include an extended release composite film comprising: a drug-loaded membrane having a plurality of drug particles embedded in a polymer matrix; a first porous coating contacting the drug-loaded polymeric membrane and forming a first interface; and a second porous coating contacting the drug-loaded polymeric membrane and forming a second interface, wherein, the drug-loaded membrane has a thickness of about 25-500 microns, and at least 90% of the plurality of the drug particles have a diameter in the range of 10-80 microns, and wherein the drug-loaded membrane comprises 20-70% by volume of the plurality of the drug particles in the drug-loaded membrane.

Another embodiment provides an extended release composite film comprising: a drug-loaded membrane having a plurality of drug particles embedded in a polymer matrix; a first porous coating contacting the drug-loaded polymeric membrane and forming a first interface; and a second porous coating contacting the drug-loaded polymeric membrane and forming a second interface, wherein, each drug particle is in contact with at least one other drug particle, and wherein at least one drug particle contacts the first interface; and at least one drug particle contacts the second interface.

In various embodiments, the drug particles comprise one or more antibiotics.

In various embodiments, the drug particles are completely released or nearly complete released (at least 90%) in a period of 5-14 days, or 7-14 days or 7-10 days.

Also provided is a method of providing antibiotic prophylaxis comprising inserting to a surgical site an extended release composite film as described herein, wherein the drug particles comprises one or more antibiotics.

### BRIEF DESCRIPTION OF THE SEVERAL VIEWS OF THE DRAWINGS

The following figures set forth embodiments in which like reference numerals denote like parts. Embodiments are illustrated by way of example and not by way of limitation in all of the accompanying figures wherein:

FIG. 1A schematically shows an extended release composite film according to an embodiment.

FIG. 1B shows a cross sectional view of the extended release composite film.

FIG. 2A is an SEM image of a drug-loaded membrane.

FIG. 2B is an SEM image of the drug-loaded membrane after the release of the drug.

FIG. 3A is an SEM image of a porous polymer membrane formed by a polymer blend of pTMC and PEG.

FIG. 3B shows DSC profile of a blend of pTMC and PEG at a volume ratio of 60/40.

FIG. 4A is an SEM image of the porous polymer matrix after the PEG leached out.

FIG. 4B shows a DSC profile of the polymer matrix of pTMC after PEG leached out.

FIG. 5A shows respective DSC profiles of pTMC and a pTMC blended with a block copolymer.

FIG. 5B shows thermal gravimetric spectra of the two polymer matrices of FIG. 5A.

FIG. 6A shows a standard curve of the antibiotic drug metronidazole according to peak integrals of metronidazole.

FIG. 6B shows the DSC profile of metronidazole-loaded pTMC.

FIG. 6C shows the DSC profile after the drug release.

FIG. 7 shows the drug release of metronidazole from pTMC matrix and a porous pTMC/PEG matrix.

FIG. 8 shows drug release from two composite films.

FIG. 9 shows the DSC profile of the metronidazole-loaded porous polymer matrix (pTMC/PEG) before and after the drug release.

FIG. 10 shows the composite film with porous coating having the PEG porogen.

FIG. 11 shows the composite film with porous coating having the dextrose porogen.

FIG. 12 shows the cumulative release data of uncoated metronidazole-loaded membrane compared to the drug release from composite films having pTMC/PEG porous coatings.

FIG. 13 shows the cumulative release of the composite film having pTMC/dextrose porous coating, as compared to a composite film having pTMC/PEG porous coating.

FIGS. 14A and 14B show the respective DSC profiles of the pre-release composite film having pTMC/PEG porous coatings as compared to the post-release composite film.

FIGS. 15A and 15B show the respective DSC profiles of the composite film having pTMC/dextrose porous coatings pre-release as compared to the post-release composite film.

FIG. 16 shows the impact on the drug release by the different porogen contents in the porous coating.

FIGS. 17A and 17B show the impact on drug release by the varying the thicknesses of the porous coatings.

FIGS. 18A and 18B show a cefazolin-loaded pTMC membrane before drug release and 12 days after the release began, respectively.

FIGS. 18C and 18D show a cefazolin-loaded pTMC and b-PLGA-co-PEG blend membrane before drug release and 12 days after the release began, respectively.

FIG. 19 shows the percentage of cefazolin release from pTMC membrane as compared to cefazolin release from polymer blends of pTMC and b-PLGA-co-PEG.

FIG. 20 shows the percentage of cefazolin release from uncoated polymer matrix as compared to cefazolin release from coated composite films.

### DETAILED DESCRIPTION

Described herein are extended-release, drug-loaded composite films capable of sustained and localized drug release over a period of time. Although many active pharmaceutical agent (API) may be suitably formulated into the drug-loaded composite films described herein, antibiotic-loaded composite film is particularly suitable as antibiotic prophylaxis for surgical procedures. Typically, the composite film can be placed at surgical site and antibiotics are released immediately in a controlled manner and the release is sustained for 5-14 days, or preferably for 7-14 days or preferably for 7-10 days. Advantageously, the composite film is formed of a biodegradable polymer or polymer blend, which fully degrades after the drug release is completed.

The drug release mechanism is based, in part, on dissolution of drug particles (e.g., drug crystals) and the subsequent diffusion of the dissolved drug from a polymer matrix. To ensure complete release of the drug particles, the drug loading is above certain threshold level (i.e., a percolation threshold) whereby the drug particles are continuously in contact with one another (i.e., each particle is in contact with at least one other particle). Drug particles that are not in contact with any other drug particles tend to be trapped in the polymer matrix and will not release until the polymer matrix erodes or degrades. For drug particles above the percolation threshold, the process of dissolution and diffusion creates a porous network throughout the polymer matrix, thus enabling complete release of the drug particles over a period of time.

In addition to the dissolution characteristics of the drug particles, other factors also impact the rate or period of release. In particular, the polymer matrix may further comprise porogens that create additional porosity within the polymer matrix. The polymer matrix may also comprise hydrophilic block hydrophobic polymer or copolymer that facilitates water uptake and hydration of the embedded drug particles. Moreover, the release period may be further controlled and extended by coating the drug-loaded polymer matrix with additional layers of porous polymer coatings.

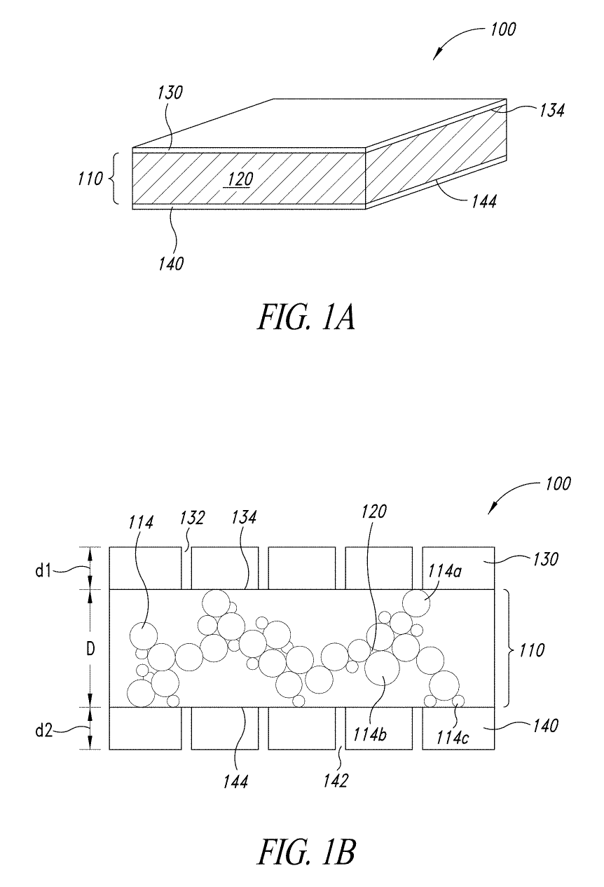

FIG. 1A schematically shows an extended release composite film according to an embodiment. FIG. 1B shows a cross sectional view. As shown in FIG. 1A, an extended release composite film (100) comprises a drug-loaded membrane (110) having a plurality of drug particles (shown as 114, 114a, 114b, 114c in FIG. 1B) embedded in a polymer matrix (120); a first porous coating (130) contacting the drug-loaded polymeric membrane (110) to provide a first interface (134); and a second porous coating (140) contacting the drug-loaded polymeric membrane (110) to provide a second interface (144), wherein, the drug-loaded membrane (110) has a thickness of, for example, about 50-500 microns, and at least 90% of the plurality of the drug particles (114) have a diameter in the range of, for example, 25-70 microns, and wherein the plurality of the drug particles occupies about 20-70% by volume in the drug-loaded membrane.

In various embodiments, the polymer matrix, the first polymer and the second polymer are biodegradable elastomers, resulting in a pliable composite film. The elastomers typically fully degrade or erode after the drug release is completed, obviating the need to remove the drug-depleted composite film.

As discussed in more detail herein, the drug-loaded membrane is typically about 50-1500 microns, or about 100-1000 microns, or about 25-500 microns, or more typically about 100-500 microns thick, the thickness being the shortest distance “D” between the first interface (134) and the second interface (144). The combined thickness of d1 and d2 of the first porous coating and the second porous coating are typically no more than 40% of D. See e.g., FIG. 1B. The overall thickness and the pliability of the extended release composite film are not particularly limited. In some embodiments, it is preferred that the composite film conforms to the site of the placement (e.g., surgical site). There is no limitation on the size (i.e., the dimension other than the thickness) of the composite film, which can be cut and shaped into appropriate sizes depending on the surgical sites. In various embodiments, all or majority (90% or more) of the drug particles are released within 5-14 days, or preferably within 7-14 days or preferably 7-10 days.

These elements are discussed in more detail below.

### Drug-Loaded Membrane

The drug-loaded membrane is the drug reservoir of the extended-release composite film, also referred to as the core of the composite film. Advantageously, the drug-loaded membrane is constructed to allow for sustained and complete release (100%) or near complete release (at least 90%) of the loaded drug. At certain drug-loading level (e.g., 20-70% v/v), as shown in FIG. 1B, each of the plurality of drug particles (114) is in contact with at least one other drug particle, and at least one drug particle (114a) reaches the first interface (134) and at least one drug particle (114c) reaches the second interface (144). The distribution of the drug particles can be random and islands of particles may form.

At this level of particle distribution, the drug release is immediate upon contacting an aqueous medium (e.g., fluid at a surgical site) due to the presence of the drug particles at the interfaces and the pores (132, 142) of the porous coatings (130 and 140). In FIG. 1B, the pores (132 and 142) are shown schematically to indicate a fluid pathway from the interface to the exterior of the composite film. The drug release is also sustained as the drug particles (114b) within the polymer matrix dissolve and diffuse out of the polymer matrix through a network of pores continuously created during the dissolution and diffusion.

### Drug Particles

The drug particles are solid, particulate drug substance that includes one or more active pharmaceutical ingredients (API). Although any API is suitable for the present disclosure, antibiotics drugs are particularly suited because a large dose of the antibiotic drugs can be loaded in the composite film and the localized dissolution-controlled release is substantially steady during the sustained-release period.

Thus, in various embodiments, the drug particles are particles of antibiotics such as cefazolin, vancomycin and metronidazole. Cefazolin is the mainstay antibiotic used to treat Gram positive bugs in surgical site wound infections. Patients getting cardiac valve replacement surgery are treated with vancomycin in addition to cefazolin. In GI surgeries, metronidazole is used widely to treat Gram negative bacteria. Other antibiotics may include for example, clindamycin, rifampin, cefoxitin, gentamicin, ampicillin, sulbactam, fluconazole, aztreonam, amoxicillin/clavulanate, ampicillin/culbactam, ceftriaxone, cephazolin, ciprofloxacin, piperacillin/tazobactam, trimethoprim, sulfamethoxazole, levofloxacin, penicillin and the like.

In various other embodiments, the drug or API may be an anticancer agent, or an analgesic such as a local anesthetic.

In other embodiments, the drug or API may be a combination of antibiotic and an analgesic agent, or a combination of an anti-cancer agent and an analgesic agent.

The dissolution rate depends, in part, on the solubility of the drug or API. In various embodiments, the drug or API has solubility in the range of the 0.05 mg/ml to 100 mg/ml.

The drug particles are typically, though not necessarily, crystalline. Crystalline drug particles are also referred to as drug crystals. Commercial drug particles may already be in a crystalline form. In some embodiments, the drug crystals are products of recrystallization, which process yields pure drug that can be easily micronized and sized.

In other embodiments, the drug particles are solid particles compounded or formulated by combining an API with one or more soluble, pharmaceutically inert ingredients. The pharmaceutically inert ingredients may act as release modulators that facilitate the dissolution or fluid uptake, or participate in the pore-forming process. The pharmaceutically inert ingredients may also act as a filler, a binder, a matrix-forming or bulking agent that modulate the size of the drug particles or the amount of the drug loading, in addition to or instead of modulating the release. Examples of the pharmaceutically inert ingredients include, without limitation, lactose, sucrose, hydroxypropyl methylcellulose (HPMC), or other hydrophilic components.

The size or diameter of a given drug particle refers to its longest dimension. In various embodiments, all or nearly all (more than 90%) of the drug particles or drug crystals have diameters of 80 microns or less. In other embodiments, all of the drug particles or drug crystals have diameters of 50 microns or less. In other embodiments, the drug particles are no smaller than 20 microns, or not smaller than 10 microns. Small crystals (smaller than 20 or 10 microns) do not readily make contacts with each other, and will be trapped in the polymer matrix. Thus, in certain embodiments, less than 10% of the drug particles in the polymer matrix are smaller than 20 microns. In other embodiments, less than 5% of the drug particles in the polymer matrix are smaller than 20 microns. In certain embodiments, the drug particles have diameters in the range of 10-80 microns.

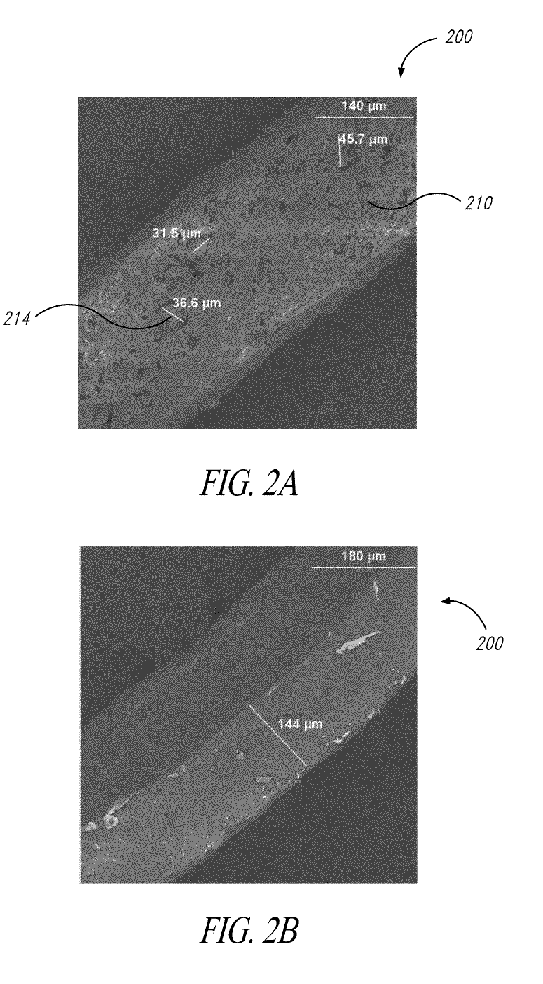

A narrow particle size distribution is preferred. Typically, crystalized drug (e.g., metronidazole) may be micronized (e.g., using a pestle and mortar) and sieved to provide drug crystals of a narrow size distribution. Size distribution may be expressed as mean diameter and standard deviation, or by the percentage particles within certain range. In certain embodiments, at least 90% of the drug particles loaded in a polymer matrix have diameters of 10-80 microns or more preferably 20-50 microns. In other embodiments, the plurality of the drug particles has a mean diameter of 40 microns and standard deviation of less than 50% of the mean diameter. The size of drug particles could be engineered based on the thickness of the membrane. The diameters of the drug particle or drug crystal may be controlled by sieving using various mesh sizes and can be directly measured with the aid of scanning electron microscope (SEM). FIG. 2A is an SEM image of a drug-loaded membrane (200) having a plurality of drug particles (214) embedded in a polymer matrix (210). No porous coatings are present. The drug particles are shown to be within the range of 20-50 microns in diameters.

The drug particles are loaded in the polymer matrix in an amount sufficient to enable complete release (100%) or near complete release (at least 90%) of all of the drug particles. FIG. 2B is an SEM image of the drug loaded membrane (200) of FIG. 2A after the drug is fully released. To avoid trapping drug particles within the polymer matrix, the amount of the drug particles should, at a minimum, reach a percolation threshold, meaning that each drug particle is in contact with at least one other drug particle, and at least one drug particle reaches the outer surface of the polymer matrix (see, the interface between the drug-loaded membrane and the porous coatings of FIG. 1B). The volume occupied by the drug particles corresponds to the overall porosity created in the polymer matrix after the drug particles are released.

Thus, in various embodiments, the volume percentage of the drug particles is about 20-70% of the drug-loaded membrane. Volume percentage (or “v/v %”) refers to the percentage of the total volume of all the drug particles within the total volume of the drug-loaded membrane. In various embodiments, the volume percentage of the drug particles is about 20-60%, 20-50%, 20-40% of the drug-loaded membrane.

As used herein, “about” refers to a range of values ±30% of a specified value. For example, the phrase “about 10 micrometers” includes a range of ±30% of 10 micrometers, namely, 7-13 micrometers.

### Polymer Matrix

The polymer matrix is the scaffold in which the drug particles are incorporated. The polymer matrix comprises one or more biodegradable polymers. Examples of the polymers include, without limitations, polytrimethylene carbonate, poly D,L-lactide, polylactide glycolide, i.e., poly(lactic-co-glycolic acid) (PLGA), polycaprolactoe, polyadibic anhydride, polysebacic acid, polyvinyl alcohol, and copolymers or terpolymers thereof.

Elastomers are particularly suitable due to their rubbery and pliant characteristics. Membranes or films formed of elastomers readily conform to the sites of administration, such as surgical sites. Suitable elastomers typically have a glass transition (Tg) temperature below 37° C.

In certain embodiments, the polymer matrix is formed of polytrimethylene carbonate or a block copolymer comprising the same. Polytrimethylene carbonate (pTMC) has a Tg of −12.57° C. and degrades by surface erosion via oxidation and/or enzyme hydrolysis without producing acid degradation products. The degradation time of pTMC is a function to its molecular weight. For example, a pTMC film with a molecular weight of 150+/−50 kDa implanted subcutaneously in rats would degrade within one month.

Although the polymer matrix degrades or erodes in the presence of endogenous enzymes, the degradation period of the suitable polymers is typically much longer than the drug release period. Thus, the release of the drug particles from the polymer matrix is predominately due to diffusion of the dissolved drug through a porous network within the polymer matrix, rather than erosion of the polymer matrix.

In some embodiments, the polymer matrix only becomes porous as the drug particles dissolve and diffuse. In other embodiments, the polymer matrix may additionally comprise a porogen, which is a substance that creates pores within the polymer matrix but is pharmaceutically inactive. Porogens may be water-soluble particles of salt or sugar (e.g., dextrose), which dissolve and diffuse like the drug particles but are themselves pharmaceutically inactive.

The presence of the particulate porogens can modulate the amount of the drug-loading and consequently the dosing and the release period. When a porogen is present, the combined volume of the porogen and the drug particles should be in the range of 20-70% (v/v) of the total volume of the drug-loaded membrane to ensure that the particles reach the percolation threshold.

In other embodiments, the polymer matrix may be naturally porous, which means that it is innately porous independent of the pores created by the dissolution and diffusion of drug particles or particulate porogens. Naturally porous polymer matrix may be prepared by blending a biodegradable polymer (e.g., pTMC) with a water-soluble polymer such as polyethylene glycol (PEG). Typically, PEG has a low molecular weight of 20 kDa or less. As used herein, “blending” means that two or more polymers or substances are physically combined, without the formation of chemical bonds.

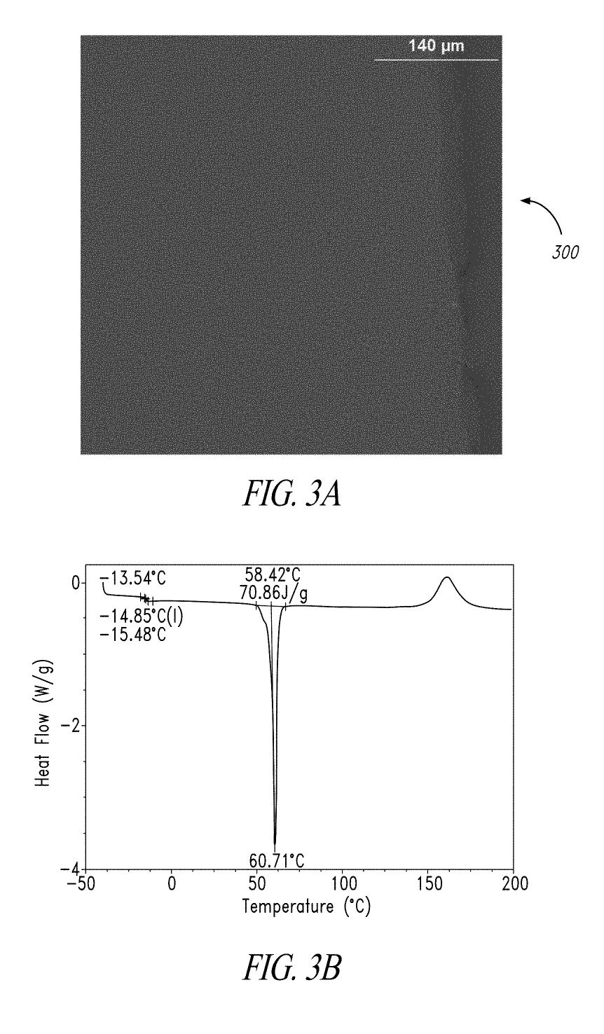

FIG. 3A is an SEM image of a porous polymer matrix or membrane (300) formed by a polymer blend of pTMC and PEG. No drug particles are present. The membrane can be formed by co-dissolving the polymer blend and cast in a mold. As shown in FIG. 3A, the membrane is porous with pores distributed throughout the membrane.

The porosity, i.e., the volume percentage of all the pores within in the membrane, can be measured using gas-based techniques, gravimetric or fluid resaturation techniques. More specifically, thermal analysis using DSC may be used to evaluate the porosity and pore connectivity of the membrane formed of a blend of pTMC and PEG. FIG. 3B shows DSC profile of a blend of pTMC and PEG at a volume ratio of 60/40. Peaks in the DSC profiles correspond to the melting point (Tm) of PEG (60.71° C.) and pTMC (−14.48˜−15.48° C.).

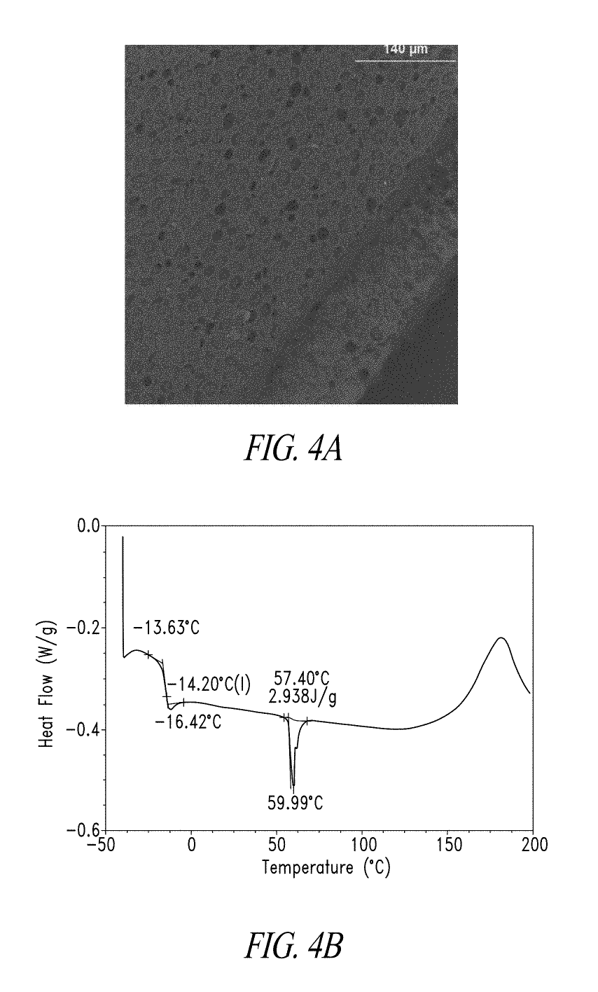

When hydrated in an aqueous medium, the water soluble PEG leaches out of the polymer matrix and creates additional porous network within the polymer matrix. Thus, like particulate porogens, PEG also acts as a porogen (i.e., a polymeric porogen). FIG. 4A is an SEM image of the porous polymer matrix (300) after the PEG leached out. As shown, the pores in FIG. 4A have enlarged compared to those pores in FIG. 3A.

FIG. 4B shows a DSC profile of the polymer matrix of pTMC after PEG is leached. As shown, the amount of PEG has substantially reduced and the percentage of leached PEG can be ascertained by the areas of respective peaks of PEG and pTMC before and after hydration.

The reduction in enthalpy of endotherm of PEG (T_{m,peak}=60.71° C.) as a result of hydration may be used as indication of the percent PEG removed, as well as the level of porosity and pore connectivity. As shown in Table 1, in a polymer blend of pTMC and PEG in a volume ratio of 60/40, nearly all of the PEG leached out after hydration; whereas in polymer blends having less PEG contents, some PEG remained trapped.

TABLE 1

| | ΔH non- | ΔH | Percent |
|---|---|---|---|
| PEG | hydrated | hydrated | PEG |
| content (v/v) | (J/g) | (J/g) | leached (%) |
| 10 | 17.66 | 7.77 | 55.96 |
| 20 | 32.43 | 4.57 | 85.91 |
| 40 | 70.86 | 2.94 | 95.85 |

In further embodiments, pTMC is blended with a block copolymer comprising at least two biodegradable polymers. Typically, one of the biodegradable polymers is a hydrophilic polymer (e.g., PEG). The other biodegradable polymer may be a hydrophobic polymer, such as poly(D,L lactic acid) (PLDDA). It may itself be a copolymer such as PLGA. More specifically, the block copolymer may be, for example, b- PLGA-co-PEG, b-PLDDA-co-PEG, or b-pTMC-co-PEG. The block copolymer facilitates the water uptake into the polymer matrix due to the hydrophilic block PEG. Like pTMC, the blended polymer or copolymers ultimately degrade after the drug particles have been released.

The block copolymer may be blended in at an amount from 5% (by weight) up to 80% (by weight). The amount directly impacts the rate of water uptake or diffusion into the polymer matrix, which in turn may control the rate of drug dissolution and drug release, as well as the length of the release period. In various embodiments, the block copolymer may be in an amount of 5-70%, 30-70%, 40-70%, 50-70%, or 60-70% (by weight).

The composition of the block copolymer can further fine-tune or otherwise modulate the rate of water uptake. For instance, the molar ratio or respective molecular weights of the copolymers (e.g., PEG and PLGA) may be adjusted to increase or decrease the rate of water diffusion. Typically, a higher ratio of PEG or a lower molecular weight of PEG could lead to faster water uptake or diffusion. In specifically embodiments, the block copolymer may be b-PLGA-co-PEG, wherein the PLGA block has a molecular weight of 75kDa and the PEG block has a molecular weight of 5kDa.

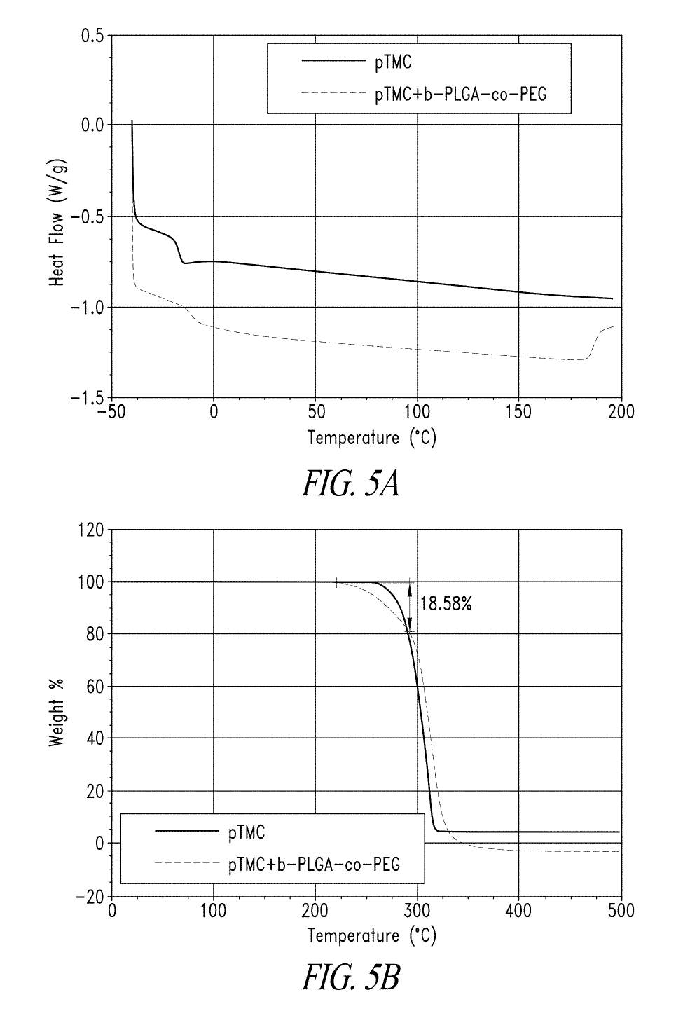

The block copolymer is typically blended well with pTMC to provide a homogeneous blend. FIG. 5A shows DSC profiles of a pTMC polymer matrix as compared to a polymer matrix of pTMC blended with b-PLGA-co-PEG (80:20 weight ratio). FIG. 5B shows thermal gravimetric spectra of the two polymer matrices of FIG. 5A. The polymer blend is shown as homogeneous.

Loading the Drug Particles into the Polymer Matrix

As discussed herein, the polymer matrix (whether a single polymer or a polymer blend or a copolymer) is a reservoir hosting the drug particles. The drug particles are loaded into the polymer matrix at a certain volume percentage to ensure that during release, little (less than 10%) or none of the loaded drug particles are trapped inside the polymer matrix. Additional particulate porogens may be present in the polymer matrix to modulate the drug loading amount.

The drug particle may be incorporated into the polymer matrix by first preparing a solution of the polymer and the drug particles of appropriate sizes and size distribution as discussed herein. The solvent is not particularly limited provided that it does not dissolve the drug particles. Typically, the solvent is a polar organic solvent, including for example dichloromethane or chloroform.

The polymer solution is then mixed by sonication and/or mechanical mixing to obtain a homogenous suspension. The suspension is poured into a mold of an inert material (e.g., glass). As the solvent is removed, a drug-loaded polymer membrane is formed.

In an alternative embodiment, the drug particles and a polymer or polymer blend may be mixed and extruded into a sheet or membrane, under solvent free or low-solvent conditions. The extrusion may be carried out at room temperature or an elevated temperature (hot-melt), provided that the drug particles are heat-stable under the hot-melt condition.

The thickness of the drug-loaded membrane can be controlled by the extrusion parameters, or in the case of solvent-casting, the amount or concentration of polymer/drug suspension added into a mold or by using micrometer adjustable film-casting knife. In various embodiments, the drug-loaded membrane is about 25-500 microns thick or 100-500 microns thick. In more specific embodiments, the drug-loaded membrane is about 100-300 microns thick.

As discussed herein, the plurality of the drug particles to be loaded has a narrow size distribution, e.g., at least 90% of the drug particles have diameters within the range of 20-50 microns wide. The drug loading level is about 20-70% by volume. To load the drug particles (or a combination of drug particles and particulate porogens such as sugar particles), weight percentage of the particles can be derived from the targeted volume percentage by taking into consideration of the respective densities of the drug and the polymer matrix. In various embodiments, the weight percentage of the drug particles in the drug-loaded membrane is about 25-55% (wt %).

### Porous Coatings

The drug-loaded membrane is sandwiched between two porous polymer coatings which serve to further control the drug-release, i.e., by slowing down the drug release from the drug-loaded membrane. At least some of the drug particles dispersed in the drug-loaded membrane reach the respective interfaces between the membrane and the porous coatings. The pores in the porous coating create pathways for fluid communication between the drug particles and the tissue environment (e.g., a surgical site) in which the composite film is placed.

Each of the porous coating is formed of a naturally porous polymer or a polymer blend comprising a polymer and a porogen. Similar to the porous polymer matrix, the porogen may be particulate (e.g., salt or sugar crystals) or a water-soluble polymer such as PEG, HPMC or pectin. The polymer may be the same or different from the polymer matrix of the drug-loaded membrane. In preferred embodiments, the polymer of the porous coating is the same as the polymer of the polymer matrix, thereby creating a cohesive composite film.

The porous coating may be formed by brushing or spraying a solution of the polymer blend on the surface of the drug-loaded membrane. Alternatively, the drug-loaded membrane may be dipped in a solution of the polymer blend. The thickness of the porous coating may be controlled by the concentration of the polymer in the dipping or coating solution. Dipping or coating may be also repeated to increase the thickness of the coating. In various embodiments, the thickness of the porous coating on each side of the drug-loaded membrane is no more than 20%, or no more than 10%, or no more than 5 of the thickness of the drug-loaded membrane.

The amount of the porogen in the porous coating determines the porosity, which in turn impacts the drug release. In various embodiments, the porous coating has 15-40% porosity or 25-35 v/v % porosity. In specific embodiments, the porous coating is a polymer blend of pTMC and PEG at 60/40 (v/v). In other specific embodiments, the porous coating is a polymer blend of pTMC and dextrose particles.

### Drug Release From the Composite Film

The composite films described herein are constructed to exhibit a sustained release profile uniquely suited for highly localized, extended delivery of one or more API for a period of 6 hours to up to 2-3 months.

The sustained release characteristics are determined by, among others factors, the solubility of the drug, the porosity of the polymer matrix, the amount of hydrophilic block copolymers, the amount of drug-loading in the polymeric membrane and the thickness and porosity of the porous coating. For antibiotics, various embodiments provide for sustained release for 5-14 days, or preferably 7-14 days, or preferably 7-10 days.

In vitro drug release characteristics can provide important insight and is correlatable to in vivo drug release. In vitro drug release is typically evaluated as an amount of drug release from the composite film as a function of time. The amount of drug release may be quantified using ¹H-NMR or HPLC, based on the absolute values of peak integrals.

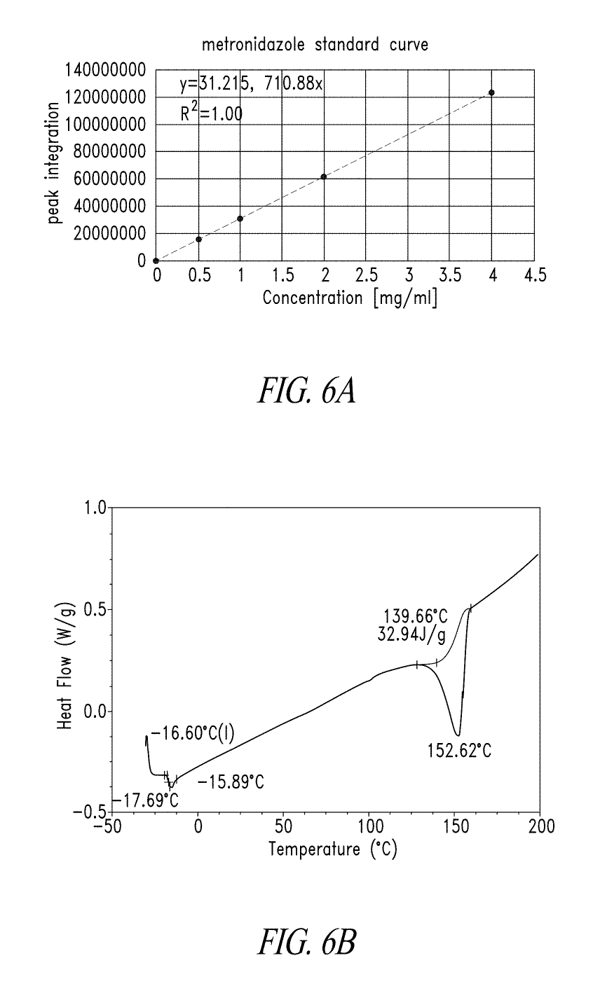

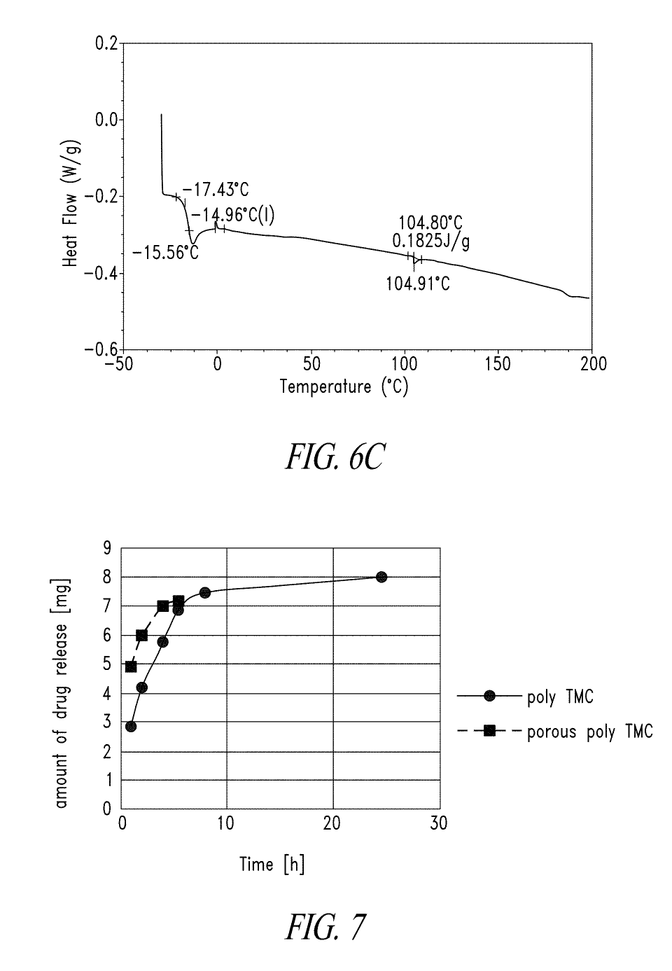

To evaluate the drug release characteristics, a standard curve of peak integral (in NMR or HPLC spectra) and drug concentration is first generated using peaks characteristic of the drug and known drug concentrations. FIG. 6A shows a standard curve of the antibiotic drug metronidazole according to peak integrals of the ¹H-NMR peaks of metronidazole.

A sample of the released drug in an aqueous release medium is taken at a given time interval and subject to ¹H-NMR or HPLC for quantification. The release amount can be ascertained by comparing the peak integral to the standard curve. Release was carried out in deuterated water and phosphate-buffered saline (PBS) buffer.

Alternatively, DSC profiles and peak intensity may also be used to quantify the drug release. FIG. 6B shows the DSC profile of metronidazole-loaded pTMC. The profile shows an intense signal corresponds to metronidazole at Tm of 155° C. FIG. 6C shows the DSC profile after the drug release was completed, as evidenced by the absence of the peak at 155° C.

The drug release is typically faster in a naturally porous polymer matrix than from a non-porous polymer matrix. FIG. 7 shows the drug release of metronidazole from pTMC matrix as compared to the release from a porous pTMC/PEG matrix. As shown, the antibiotic drug release from the porous polymer matrix (pTMC/PEG) was much feaster and reached near completion within 5 hours. In contrast, the drug release from pTMC matrix was slower and took longer (about 10-20 hours) to fully release.

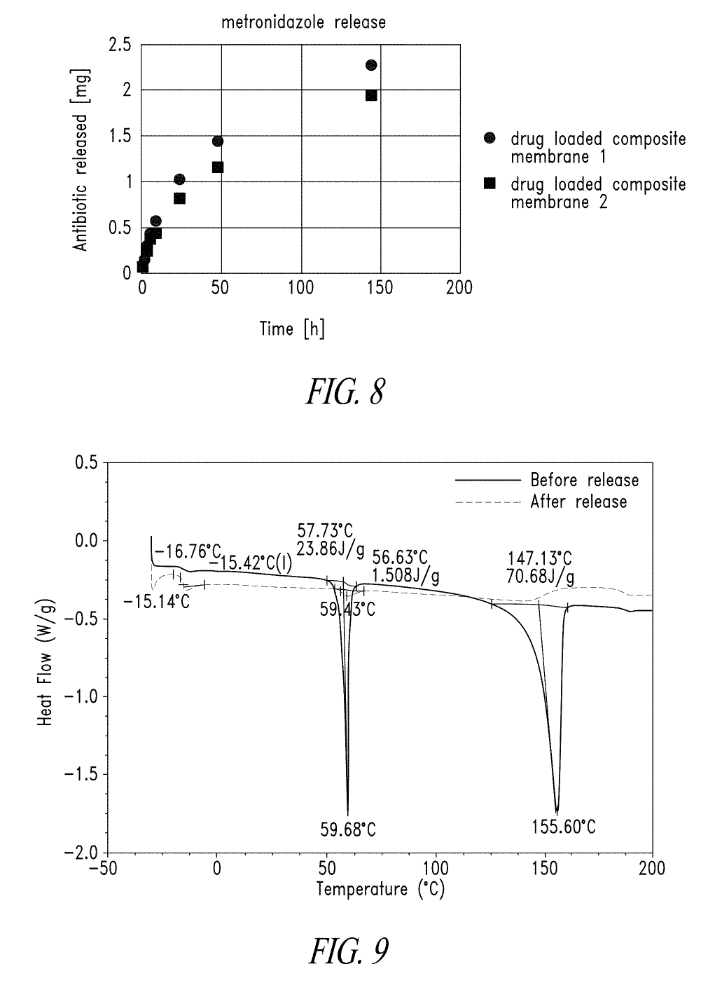

The drug release from the drug-loaded membrane is typically significantly impeded by the porous coatings. Compared to the drug release from drug-loaded membrane without porous coating (see, e.g., FIG. 7), the composite film disclosed herein is capable of releasing the drug over a period of days. FIG. 8 shows drug release from two composite films and sustained release was achieved for about 150-240 hours (compared to 10-20 hours without the porous coatings).

### Methods of Using and Routes of Administration

The drug-loaded composite film described herein is suitable for placing in a tissue environment where highly localized and extended release of the drug is beneficial. In a particularly preferred embodiment, the composite film is loaded with crystals of one or more antibiotics and is used for surgical prophylaxis. In particular, the composite film is placed at a surgical site post-surgery and prior to the closure of the incision. Upon hydration by the fluid at the surgical site, immediate release of the antibiotics takes place and the release is sustainable for 7-10 days, a critical period surgical prophylactic use of antibiotics.

### EXAMPLES

### Example 1

### Drug-Loaded Membranes

Metronidazole was recrystallized and sized using a 50 μm sieve. The resulting metronidazole crystals were suspended in dichloromethane with pTMC (MW=150+/−50 kDa) and cast into a film.

The loading level by weight of metronidazole was 33.3% (i.e., weight percentage of metronidazole of the total weight of metronidazole and pTMC). Given the density of metronidazole (1.45 g/cm³) and pTMC (1.24 g/cm³), the corresponding volume percentage of the metronidazole crystals was 30% v/v.

### Example 2

### Drug-Loaded Porous Membranes

Metronidazole-loaded naturally porous membrane was also prepared in a similar process as Example 1. In particular, metronidazole crystals were suspended in 60/40 (v/v) of pTMC and PEG (8 kDa) and cast into a porous film. FIG. 9 shows the DSC profile of the metronidazole-loaded porous polymer matrix (pTMC/PEG) before and after the drug release. Complete or near complete drug release was evidenced by the absence of the peak characteristic of metronidazole (Tm=155° C.). In addition, the peak characteristic of PEG (Tm=60° C.) also disappeared after the release, indicating that PEG also leached from the polymer matrix.

### Example 3

### Composite Film with Peg Porogen

The drug-loaded membrane of Example 1 was coated with porous coatings. A solution of pTMC and PEG (40/60 by weight) was prepared to form a porous coating of 40 wt % of PEG. The porous coating was formed by brushing the drug-loaded membrane with the pTMC/PEG solution or by dipping the drug-loaded membrane into the polymer solution. The total thickness of the composite film is about 250 μm, including the porous coatings at about 5 to 10 μm thick each (for a total of 10% increase in thickness after dipping).

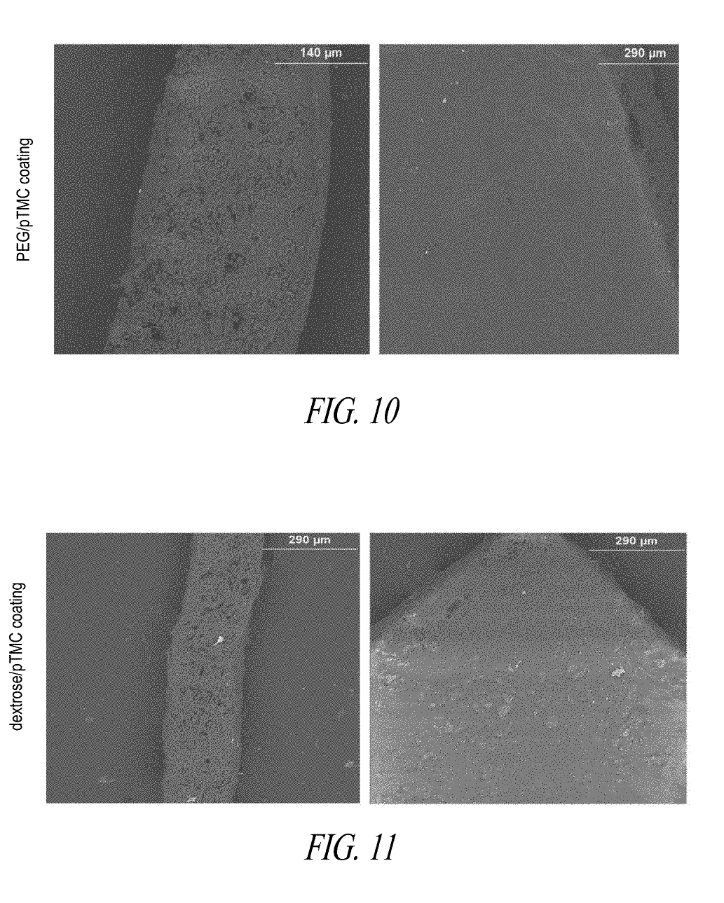

FIG. 10 shows the composite film with porous coating having the PEG porogen. The left panel is a view of the cross section of the composite film (i.e., the thickness dimension); whereas the right panel is the coating surface of the composite film. It could be observed that the coating surface had a smooth appearance due to the homogeneity of the polymer blend of pTMC and PEG (i.e., the polymer chains of pTMC and PEG interspersed and formed a single phase).

### Example 4

### Composite Film with Dextrose Porogen

The drug-loaded membrane of Example 1 was coated with porous coatings. A solution of pTMC and dextrose crystals was prepared to form a porous coating of 33.3 wt % of the dextrose porogen. The porous coating was formed by brushing the drug-loaded membrane with the pTMC/dextrose solution or by dipping the drug-loaded membrane into the polymer solution. The total thickness of the composite film was 175 μm. Each of the porous coatings were about 5-10 μm thick.

FIG. 11 shows the composite film with porous coating having the dextrose porogen. The left panel is a view of the cross section of the composite film (i.e., the thickness dimension); whereas the right panel is the coating surface of the composite film. It could be observed that the coating surface had a rough (less smooth than the PEG/pTMC coating) appearance due to the heterogeneity of the polymer blend of pTMC and dextrose particle.

### Example 5

### Quantification of Drug Release

Release experiments were performed in deuterated water in 2 ml Eppendorf tubes, tubes were located in a shaker (Thermo Fisher) set at 37° C. and agitated at 650 rpm. Release media was replaced frequently to avoid saturation.

A 2 ml-sample of the release medium was taken out at 1, 2, 4, 7, 24, 48, 72, 144 and 240 hours. The amount of the drug release at each time interval was measured by ¹H NMR; and the peak integrals were used to ascertain the amount of drug by comparing to a standard curve (e.g., FIG. 5).

Quantification by HPLC was also used to evaluate the drug release in PBS buffer as a function of time, and produced similar results.

### Example 6

### Drug Release from Ptmc/Peg Coated Composite Film

Cumulative release of metronidazole from a metronidazole-loaded composite film having a metronidazole/pTMC membrane coated with PEG/pTMC porous coatings was evaluated by ¹H NMR. Metronidazole-loaded pTMC membrane samples were cut from a larger membrane that contained a theoretical loading of 33.3 w/w % metronidazole. Both uncoated and coated TMC membranes were sectioned from the same membrane. The porous PEG/pTMC coating layer contained 40 w/w % of PEG. The coating layers did not contain any metronidazole. The coating layer was formed either by dip coating or brushing.

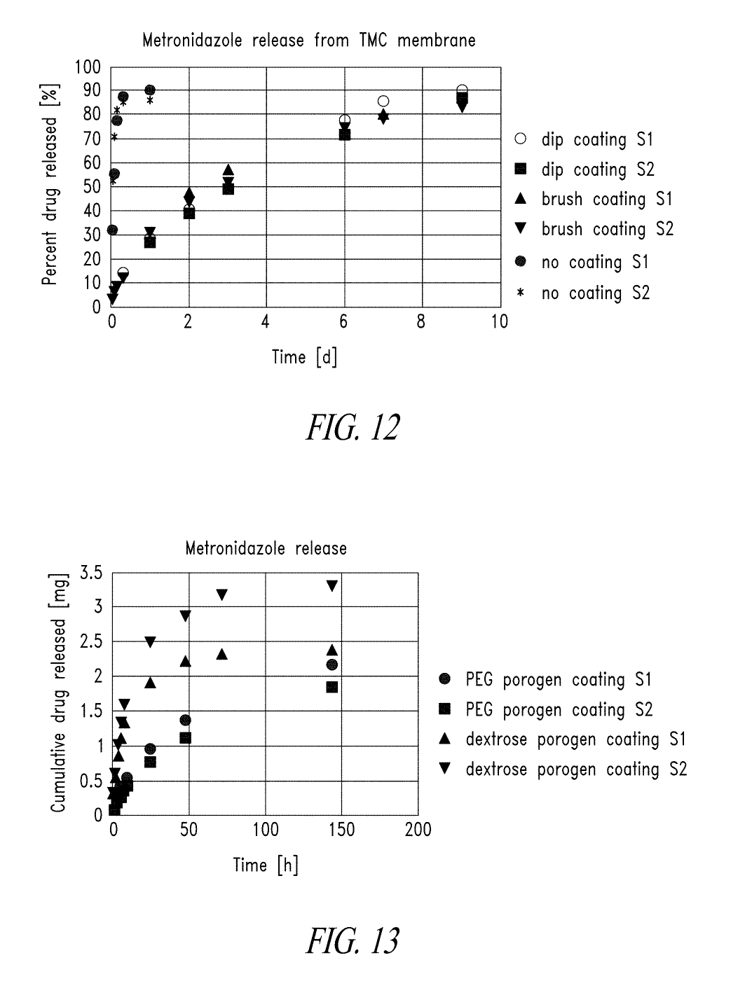

FIG. 12 shows the cumulative release data of two samples (S1 and S2) of uncoated metronidazole-loaded membrane, dipped coated metronidazole-loaded composite film and brushed coated metronidazole-loaded composite film, released in PBS at pH 7.4. As shown, coated composite films provide much more extended release of metronidazole (over 7 days) compared to less than 10 hours for the uncoated metronidazole-loaded membrane.

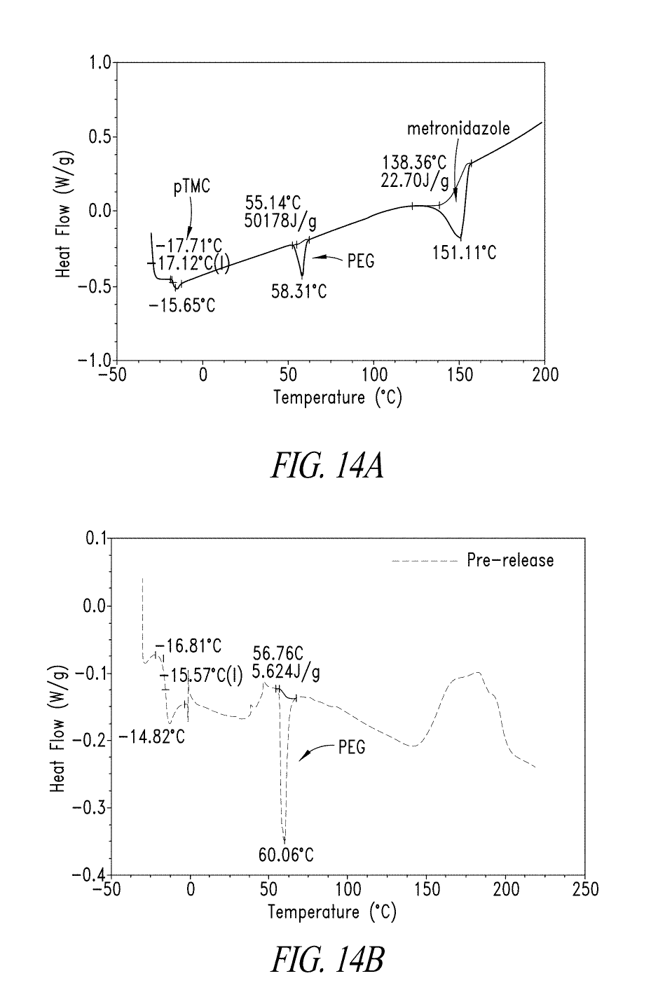

FIGS. 14A and 14B show the respective DSC profiles of the composite film pre-hydration and drug release compared to post-release. As shown, the peaks corresponding to PEG (in the coating layer) and the drug in the membrane disappeared after release.

### Example 7

### Drug Release from Ptmc/Dextrose Coated Composite Film

Cumulative release of metronidazole from a metronidazole-loaded composite film having a metronidazole/pTMC membrane coated with pTMC/dextrose porous coatings was evaluated by ¹H NMR. FIG. 13 shows the cumulative release of the composite film, as compared to the composite film having pTMC/PEG porous coating. As shown, the pTMC/dextrose coating slowed down the release to a lesser extent than the composite having the pTMC/PEG coatings.

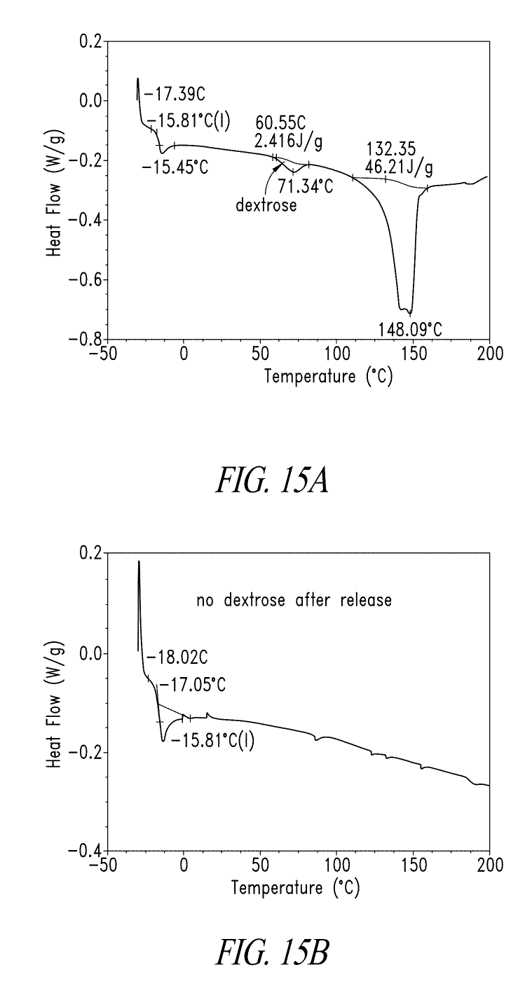

FIGS. 15A and 15B show the respective DSC profiles of the composite film pre-hydration and drug release compared to post-release. As shown, the peaks corresponding to dextrose (in the coating layer) and the drug in the membrane disappeared after release.

### Example 8

### Drug Release Impacted by Porogen Contents

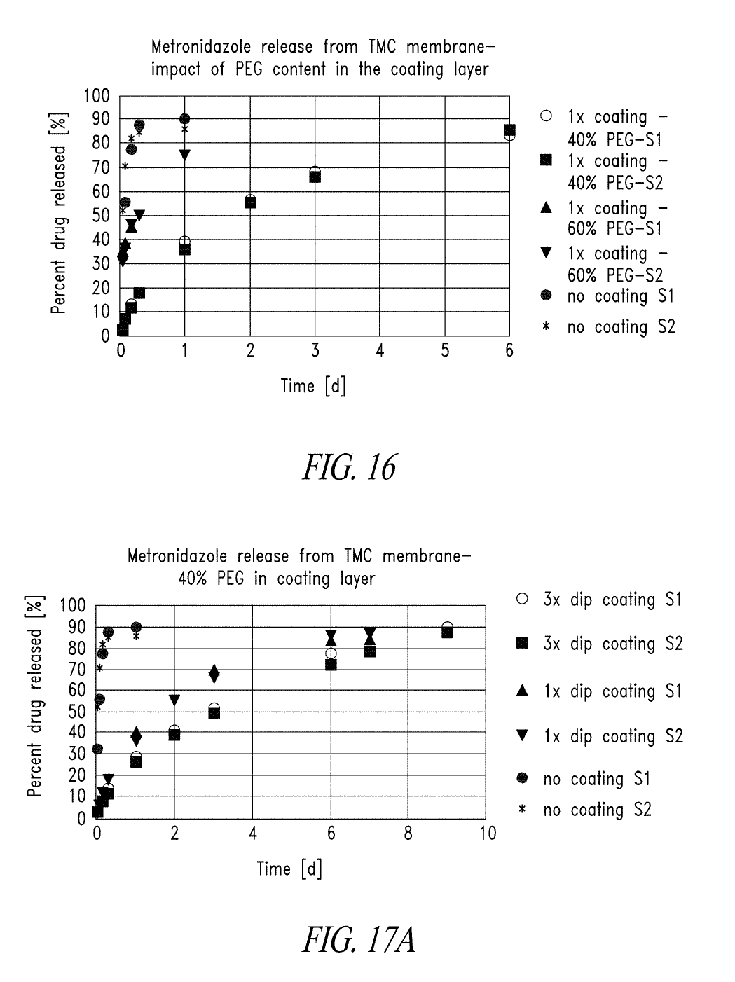

FIG. 16 shows that drug release can be impacted by the amount of porogens present in the porous coating. Consistent with the results shown in Example 6, composite films having porous coatings (40% PEG or 60% PEG) exhibited slower release when compared to uncoated drug-loaded membranes. FIG. 16 further shows that the release from composite films having different coatings can differ significantly depending on the amounts of the porogens in the porous coating. More specifically, composite films having porous coatings of 40% PEG showed much slower release as compared to that of the composite films having porous coatings of 60% PEG. The result indicates that at higher content of PEG (60%) and higher porosity, the porous coating impedes the drug release to a lesser degree than the porous coating of lower PEG content (40%).

### Example 9

### Drug Release Impacted by the Thickness of the Porous Coatings

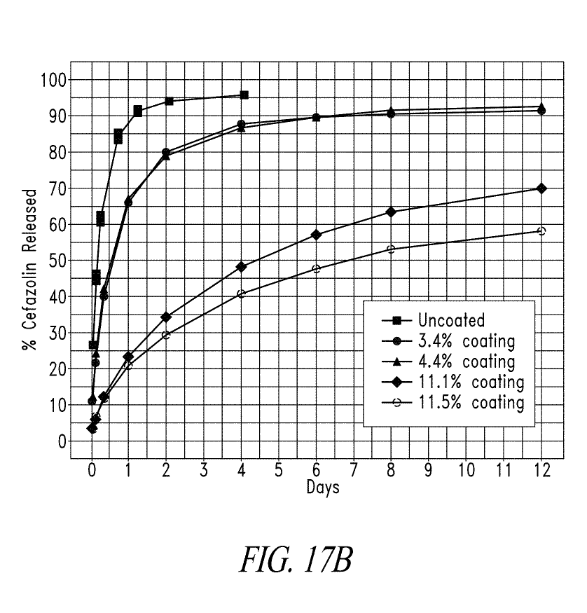

FIG. 17A shows that, at the same porogen percentage content (40%), the thickness of the porous coating can impact the drug release. In particular, the thicker the porous coating (3× dip coating), the more the porous coatings impeded the drug release.

FIG. 17B shows a similar trend for porous coatings formed by pTMC blended with 30% PEG. The percentages shown are the percentages of the coating thickness in reference to the thickness of the drug-loaded membrane, wherein 0% means that the drug-loaded membrane was uncoated. Similarly to the results shown in FIG. 17A, the drug release slowed down as the coating thickness increased.

### Example 10

### Impact of Porosity of Polymer Matrix on Drug Release in Cefazolin-Loaded Membranes

Cefazolin (1.1 g) was recrystallized from a mixture of acetone and water (50:50 v/v, 100 ml) in a beaker covered by an aluminum foil. Cefazolin was first fully dissolved under stirring at 40° C. Upon return to room temperature, the solvent was allowed to evaporate slowly through holes made in the aluminum foil. After 2-3 days, the recrystallized cefazolin crystals were separated, dried, milled and passed through 53 or 75 μm sieves.

The recrystallized cefazolin was loaded in two types of polymer matrices to determine the impact of membrane polymer composition on the drug release. More specifically, following sieving through <75 μm sieves, cefazolin crystals was loaded at 33.3% w/w level in (1) pTMC polymer matrix and (2) 80:20 (by weight) polymer blend of pTMC and b-PLGA-co-PEG (MW of PLGA=75 kDa, MW of PEG=5 kDa).

The two cefazolin-loaded membranes were tested for drug release in a PBS buffer for 12 days.

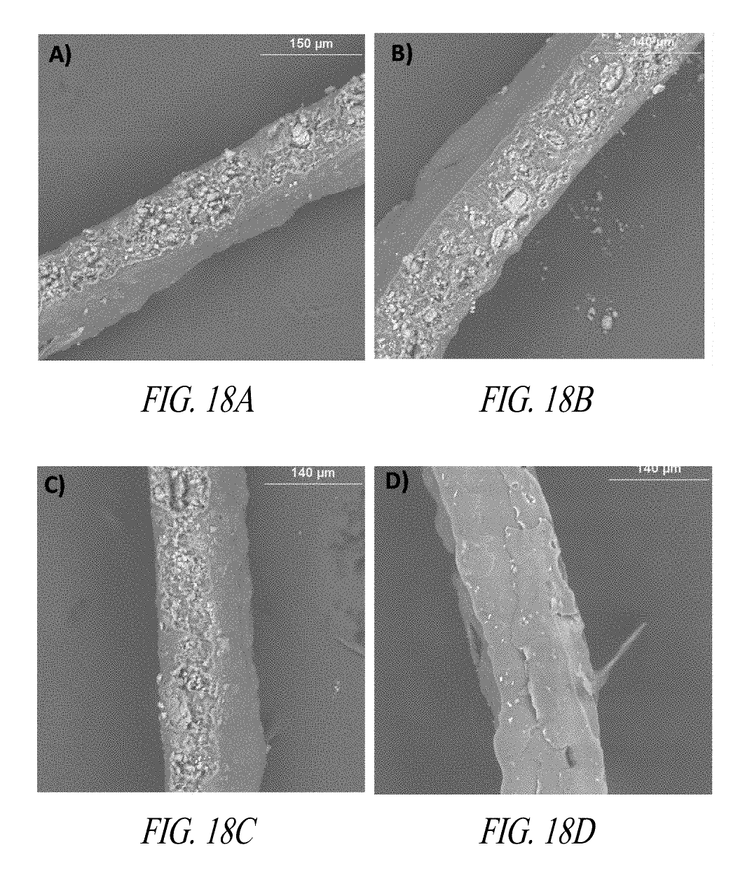

FIG. 18(A) and 18(B) show a cefazolin-loaded pTMC membrane before drug release and 12 days later, respectively. As shown, a majority of the drug crystals remained trapped in the membrane.

FIG. 18(C) and 18(D) show a cefazolin-loaded pTMC and b-PLGA-co-PEG blend membrane before drug release and 12 days later, respectively. As shown, after 12 days, nearly all of the drug crystals had been released, leaving little or no trapped crystals in the membrane.

FIG. 19 shows the percentage of cefazolin release from the pTMC membrane as compared to cefazolin release from polymer blend of pTMC and b-PLGA-co-PEG (two samples). FIG. 19 quantitatively demonstrates that, despite having the same drug loading (33% w/w), more than 95% of cefazolin had been released from the polymer blend matrix after 12 days; whereas more than half of the cefazolin crystals remained trapped in the pTMC matrix after the same period (12 days).

Thus, it is demonstrated that polymer matrix comprising hydrophilic, water-soluble or biodegradable polymer or block copolymer can significantly increase the rate and extent of drug release. The block copolymer (such as PEG or b-PLGA-co-PEG, b-pTMC-co-PEG, b-PLLDA-co-PEG) is capable of improving water-uptake and fluid diffusion within the polymer matrix, resulting in complete or near complete drug release with little trapped drug crystals.

### Example 11

### Cefazolin-Loaded Composite Film with Porous Coatings

The cefazolin-loaded membrane of Example 10 (polymer blend of pTMC and b-PLGA-co-PEG at 80:20 weight ratio) was further coated on both sides of the membrane by a thin layer of pTMC and b-PLGA-co-PEG polymer blend. The coating comprised the same polymer blend as the polymer matrix, with an additional amount of PEG porogen. Three types of coating blend were prepared with different PEG porogen contents (30%, 50% and 70%). Each coating was no more than 10% of the weight of the drug-loaded polymer matrix.

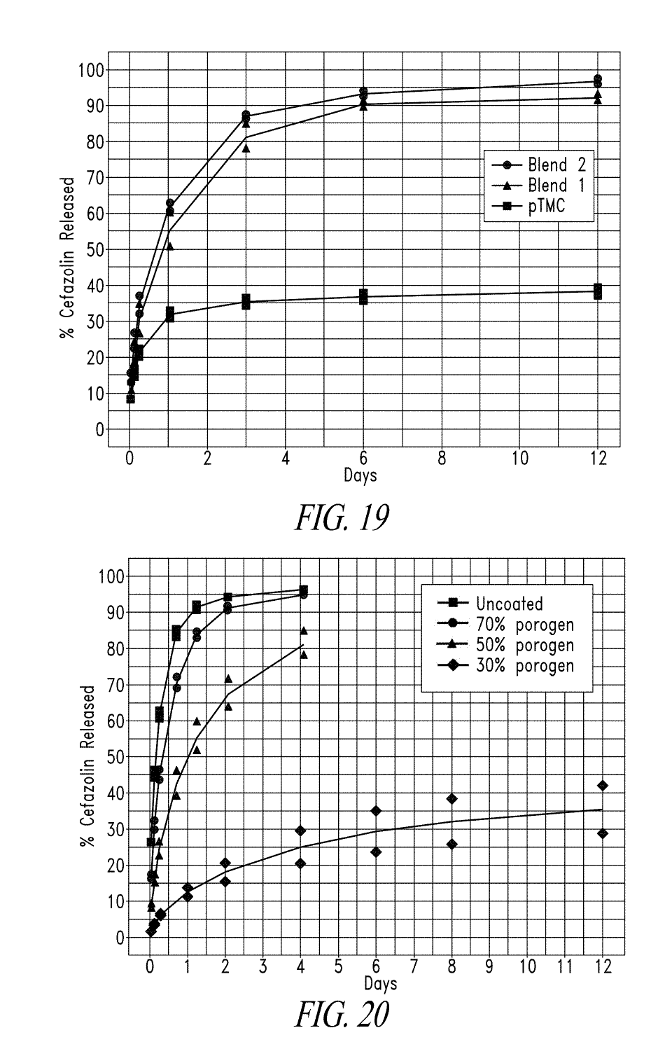

The drug dissolution tests were carried out and the results are shown in FIG. 20. As shown, drug release from the uncoated polymer matrix was rapid initially and quickly reached near complete release. In contrast, drug release from the coated composite film (having 70% PEG in the coating) showed mild slow-down compared to the uncoated membrane, indicating that the coatings were impeding the drug release from the membrane. For the composite film that has the lowest amount of PEG porogen (30%), the drug release slowed down significantly, indicating that a lesser amount of porogen further impedes the drug diffusion. It is thus demonstrated that the polymer coatings on the drug-loaded membrane are capable of slowing down the drug release. The amount of porogen within the polymer coating can be adjusted to further modulate the rate of the drug release.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

We claim:

1. An extended release composite film comprising:
a drug-loaded membrane having a plurality of drug particles embedded in a polymer matrix;
a first porous coating contacting the drug-loaded polymeric membrane and forming a first interface; and
a second porous coating contacting the drug-loaded polymeric membrane and forming a second interface,
wherein, the drug-loaded membrane has a thickness of about 25-500 microns, and at least 90% of the plurality of the drug particles have a diameter in the range of 10-80 microns, and wherein the drug-loaded membrane comprises 20-70% by volume of the plurality of the drug particles in the drug-loaded membrane.

2. The extended release composite film of claim 1 wherein at least one drug particle contacts the first interface; and at least one drug particle contacts the second interface.

3. The extended release composite film of claim 1 wherein the first porous coating comprise a first polymer and a first porogen; and the second porous coating comprises a second polymer and a second porogen.

4. The extended release composite film of claim 1 wherein the polymer matrix, the first polymer and the second polymer are the same or different and independently an elastomer.

5. The extended release composite film of claim 1 wherein the polymer matrix, the first polymer and the second polymer are the same or different and independently selected from the group consisting of polytrimethylene carbonate, poly D,L-lactide, polylactide glycolide, polycaprolactoe, polyadibic anhydride, polysebacic acid, polyvinyl alcohol, copolymers and terpolymers thereof.

6. The extended release composite film of claim 1 wherein the first porogen and the second porogen is the same or different and independently a water-soluble polymer or particles of salt or sugar.

7. The extended release composite film of claim 1 wherein the polymer matrix further comprises a porogen.

8. The extended release composite film of claim 1 wherein each of the first porous coating and the second porous coating are no more than 30%, or no more than 20%, or no more than 10% of the thickness of the drug-loaded membrane.

9. The extended release composite film of claim 1 wherein each of the first porous coating and the second porous coating are no more than 30%, or no more than 20%, or no more than 10% of the weight of the drug-loaded membrane.

10. The extended release composite film of claim 1 wherein the polymer matrix of the drug-loaded membrane comprises pTMC.

11. The extended release composite film of claim 10 wherein the polymer matrix further comprise polyethylene glycol (PEG) or a block copolymer of PLGA-co-PEG, or block copolymer of PLDDA-co-PEG or block copolymer pTMC-co-PEG.

12. The extended release composite film of claim 1 wherein the first porous coating and second porous coating each comprises pTMC and PEG, wherein PEG is about 20-70% w/w of the porous coating.

13. The extended release composite film of claim 12 wherein the first porous coating and second porous coating further comprises b-PLGA-co-PEG, or b-PLLDA-co-PEG or b-PTMC-PEG.

14. The extended release composite film of claim 1 wherein the drug particles comprise one or more antibiotics.

15. The extended release composite film of claim 14 wherein the antibiotic is cefazolin, vancomycin, metronidazole, clindamycin, rifampin, cefoxitin, gentamicin, ampicillin, sulbactam, fluconazole, aztreonam, amoxicillin/clavulanate, ampicillin/culbactam, ceftriaxone, cephazolin, ciprofloxacin, levofloxacin, metronidazole, penicillin, piperacillin/tazobactam, trimethoprim/sulfamethoxazole, or a combination thereof.

16. The extended release composite film of claim 14 wherein all or at least 90% of the drug particles are released after a period of 7-10 days after placing the extended release composite film at a surgical site.

17. The extended release composite film of claim 1 wherein all or at least 90% of the drug particles are released after a period ranging from 6 hours to up to 3 months after placing extended release composite film at an implantation site.

18. The extended release composite film of claim 1 wherein the drug particles comprise one or more anti-cancer agents or analgesic, or a combination thereof.

19. An extended release composite film comprising:
a drug-loaded membrane having a plurality of drug particles embedded in a polymer matrix;
a first porous coating contacting the drug-loaded polymeric membrane and forming a first interface; and
a second porous coating contacting the drug-loaded polymeric membrane and forming a second interface,
wherein, each drug particle is in contact with at least one other drug particle, and wherein at least one drug particle contacts the first interface; and at least one drug particle contacts the second interface.

20. The extended release composite film of claim 19 wherein the drug particles have diameters ranging in 10-80 microns and the drug-loaded membrane is 25-500 microns thick.

21. The extended release composite film of claim 19 wherein all or at least 90% of the drug particles are released after a period ranging from 6 hours to 3 months of implanting the extended release composite film at implantation site.

22. The extended release composite film of claim 19 wherein all of the drug particles are released after 7-14 days of implanting the extended release composite film at a surgical site.

23. A method of providing antibiotic prophylaxis comprising: inserting to a surgical site an extended release composite film of claim 1 wherein the drug particles comprises one or more antibiotics.
